(19) United States
(12) Patent Application Publication
(10) Pub. No.: US 2005/0048105 A1
(43) Pub. Date: Mar. 3, 2005

(54) Protease inhibitor compositions for prevention and treatment of skin conditions

(76) Inventors: Amy K. McNulty, Plant City, FL (US); Tanya Jeannette Rhodes, Largo, FL (US)

Correspondence Address: CHIEF PATENT COUNSEL;SMITH & NEPHEW, INC., 1450 BROOKS ROAD, MEMPHIS, TN, 38116, US

(21) Appl. No.: 10/651,820

(22) Filed: Aug. 29, 2003

(51) Int. Cl. A61K 31/401; A61K 9/70

(52) U.S. Cl. 424/449; 514/423

(57) ABSTRACT

Treatments for diaper rash that include a protease inhibitor and a polymer with which the inhibitor is delivered. In certain embodiments, the inhibitor may comprises glycine soja protein or dipalmitoyl hydroxyproline. The polymer binds to the skin, creates a non-occlusive barrier, and is substantially resistant to being washed or rubbed off. The invention also relates to methods of preparing such formulations and to methods of treating patients in need of treatments for skin conditions associated with prolonged exposure to enzymes present in human waste.

### BACKGROUND

1. Field of the Invention

The invention relates to compositions and methods for treating skin conditions such as diaper dermatitis, erythema, or other skin conditions caused by prolonged exposure to certain enzymes.

2. Description of Related Art

Diaper rash or diaper dermatitis is an inflammation of the skin in the diaper area of newborn infants, infants, and children. While this condition is common to infants, it is certainly not limited to infants. Individuals who suffer from incontinence and use absorbent articles, colostomy patients, patients who are nonambulatory, and the elderly may also develop this condition.

It is estimated that about 10% of babies between 0 and 2 years of age will develop diaper dermatitis, and that 50-94% of incontinent adults will develop erythema. A certain portion of these individuals may even develop a stage I or II pressure ulcer.

These skin conditions are generally believed to be caused by the metabolic by-products of both urine and feces. Specifically, human feces contain endogenic proteolytic and/or lipolytic enzymes such as trypsin, chymotrypsin, elastase, and pancreatic lipase. (These enzymes are proteolytic enzymes produced in the gastrointestinal tract to digest food.) When the skin is exposed to such enzymes, the lipid-containing and protein-containing components of the skin (particularly the barrier layer, stratum corneum), can be broken down, resulting in irritation and inflammation. If the enzymes remain in contact with the skin for any appreciable period of time, they may cause irritation, predispose the skin to infection by microorganisms, contribute to skin breakdown, and cause (and/or exacerbate) various skin conditions.

The effect of fecal enzymes can be aggravated further by the presence of urine. In fact, prolonged exposure to urine itself may cause erythema and skin breakdown. Urine is normally slightly acidic. Feces of adults are normally neutral or slightly alkaline, while that of infants is slightly acidic. Some believe that it is the acidic agents which are believed to be highly irritating in the diaper area, while others believe it is primarily the alkaline components that contribute to diaper dermatitis.

In new-born babies, diapers can create a more hostile environment than that usually encountered by the skin, increasing the risk of dermatitis. The dermis is attacked and the skin is irritated and/or inflamed. There are acidic components of urine and infant stools which are not present in adult feces and which are particularly irritating. Urine consists of approximately 93-97% water and 3-7% solids which include urea, uric acid, creatine, creatinine, ammonia, and inorganic substances such as chlorides, calcium, magnesium, and phosphorous. In effect, the urea contained in the urine is broken down into ammonium hydroxide by the ureases which leads to an increase in pH.

It is believed by some that when the pH becomes basic, the enzymes produced at time of digestion, such as the proteases and the lipases of pancreatic or intestinal origin, increase their activity and thus their irritating power increases. In particular, the lipases attack the triglycerides of the sebum and provoke the release of fatty acids. The corneum, made permeable by hyperhydration, rubbing, and digestion by enzymes, loses its function as a barrier and allows other irritating molecules, such as ammonia, fecal enzymes, bacteria, and biliary salts, which might not be irritating by themselves, to migrate through the compromised skin barrier. In certain cases, an actual digestion of the epidermis of the infant's skin could occur due to the action of ureases, lipases and proteases.

Because of the problems caused by diaper dermatitis, many researchers have sought to develop treatments to combat the destructive effects caused when enzymes contained in feces and/or urine contact human skin for prolonged periods of time. Certain anti-enzyme compounds have been included in topically-applied compositions for treatment or prevention of diaper rash. For example, a water-soluble lipase inhibitor such as zinc chloride has been included in a barrier-like carrier such as polyethylene glycol. Currently available treatments for diaper rash are generally based upon the use of zinc oxide, vitamins (A, D, and D3), or some combination thereof. These active ingredients are incorporated into a cream or salve by blending them into various purified semisolid ointment bases, such as mineral oil, petrolatum, soft paraffin, lanolin, and the like. These ointments are intended to protect the skin from contact with irritants.

While such treatments may be effective for treating simple diaper rashes, severe cases of diaper rash, especially those that are often observed with incontinent adults, have proved resistant to the treatments. Additionally, these treatments tend to be greasy and messy. In order to wash them off, the skin must be rubbed or scrubbed, further irritating the skin.

Another conventional method of preventing or alleviating diaper rash is to apply powders to keep the skin dry. However, powders are also easily washed off and do not provide an effective barrier over time. In fact, many of the barriers used in conventional treatments are not effective. Most prophylactics or treatments for diaper dermatitis are barriers that rub off with exposure to fecal and urinary insult, leaving the skin exposed to such irritants.

Some researchers have studied the possibility of including substances that inhibit digestive enzymes into diaper rash creams, wipes, or into the diapers themselves. For example, proteolytic and lipolytic enzyme inhibitors react with the active site of the target enzyme and destroy the enzyme's ability to function. The notion is that delivering such enzymes to a patient's skin allows the inhibitors to function as a barrier to enzyme activity to prevent diaper rash.

One method that has been studied is to incorporate organophilic clays (that absorb and inactivate proteolytic enzymes) or protease inhibitors into diapers. The clays are intended to prevent enzymes from contacting the skin and render them incapable of causing irritation.

Other methods include applying an inhibitor in an ointment, cream, or gel to the location of the inflammation. Some inhibitors discussed by the literature for use according to this method include soybean trypsin inhibitor, aprotinin, hexamidine, p-aminobenzamidine, leupeptin, pepstatin A, chymostatin, and trypsin-chymotrypsin inhibitor. See e.g., WO 99/45973, WO 99/45974, EP 0 958 833 A1, and WO/01/52842, the contents of each of which are incorporated herein by reference.

A further method that has been explored for treating diaper dermatitis has been to add an antimicrobial protease inhibitor to a pre-moistened wipe. The antimicrobial protease inhibitor can be added directly to the wipe or directly to the liquid that moistens the wipe. Exemplary antimicrobial protease inhibitors are aromatic diamidines, such as pentamidine and/or hexamidine, and particularly hexamidine diisethionate.

The most common inhibitor discussed by the prior art literature is hexamidine diisethionate, also marketed as Elestab HP 100 (“Elestab”). Elestab is a hexamidine derivative and was initially marketed as an antimicrobial. It has also been marketed as an anti-dandruff product. Hexamidines have been shown to have enzyme inhibitory activity. However, while Elestab initially shows good inhibitory activity against trypsin, its inhibitory activity decreases rather quickly over time in a prepared solution. In other words, Elestab has a short shelf-life in solution, rendering it unworkable for purposes that require a solution that can remain stable and active over an extended period of time. Accordingly, it is highly ineffective for use in a preparation of an emulsion product, such as a lotion, cream or spray, which may remain on a shelf for many months at a time.

Without wishing to be bound to any theory, the inventor believes that this may be one reason why some prior art references incorporate the product into a diaper or product that remains dry until use—Elestab tends to be stable in the absence of water, but loses its stability when combined with water or other emulsification components. As such, Elestab is not appropriate for incorporation into an emulsified product. The inventor has recognized this fact and developed a product that provides a significant improvement over emulsified products using hexamidine.

In summary, the prior art combinations for diapers and wipes greatly add to the cost of the product and make them economically unfeasible. The compositions have a very short shelf life, requiring the care provider to maintain a constant replenishing supply on hand. Diapers also do not provide the additional protection of barrier between the skin and the proteases. By contrast, the present invention maintains its activity in emulsion, even after being stored for an extended period of time. Formulations according to certain aspects of this invention have an elegant, non-greasy feel, and will not add to clean up procedures. The emulsions of the present invention generally have a better feel than typical barrier ointments of petrolatum and zinc oxide which can be sticky, and hard to apply and remove.

There is a distinct need in the art for a formulation that acts as an effective inhibitor of fecal and urinary enzymes and that provides a superior barrier to these enzymes, preventing them from damaging the skin. There is also a need for a product that can remain stable in solution for extended periods of time. There is a further need for an inhibitor formulation that is substantially resistant to being washed off the skin. There is an even further need for an inhibitor formulation that is not greasy or messy.

### SUMMARY

Formulations described by the invention include treatments for diaper rash that include a protease inhibitor and a polymer which acts as a carrier for the inhibitor and also binds to the skin of the patient for prolonged delivery of the inhibitor. Certain embodiments incorporate an inhibitor that comprises glycine soja protein or dipalmitoyl hydroxyproline. Further embodiments incorporate a polymer that binds to the skin, creates a non-occlusive barrier, and that is substantially resistant to being washed or rubbed off. The invention also relates to methods of preparing such formulations and to methods of treating patients in need of treatments for skin conditions associated with prolonged exposure to enzymes present in human waste.

Certain aspects of the invention are directed to a protease inhibitor composition, comprising a polymer barrier and a protease inhibitor, wherein the polymer barrier binds the protease inhibitor to a patient's skin and is substantially resistant to being washed or rubbed off. In specific embodiments, the polymer barrier may be a Skinvisible®μM-series or D-series polymer. Additionally or alternatively, the protease inhibitor may be a glycine soja protein (including formulations with or without oxidoreductases) and/or a dipalmitoyl hydroxyproline composition.

Other aspects of the invention are directed to a protease inhibitor having an extended shelf life for use in treating diaper dermatitis-like skin conditions comprising a cosmetic protease inhibitor that maintains its activity in emulsion for at least about one year. In specific embodiments, the cosmetic protease inhibitor comprises a glycine soja protein or dipalmitoyl hydroxyproline.

Further aspects of the invention are directed to a method of treating diaper dermatitis comprising applying a composition comprising glycine soja protein and/or dipalmitoyl hydroxyproline to an affected area of a patient in need thereof.

Additional aspects of the invention relate to a method of formulating a diaper dermatitis treatment, comprising
(a) providing a polymeric system that can bind to a patient's skin;
(b) combining the polymeric system with glycine soja protein or dipalmitoyl hydroxyproline;
(c) adding a sacrificial protease substrate;
(d) adding a sacrificial lipase substrate;
(e) adding a pH buffering system; and
(f) forming the mixture into a cream or a lotion for topical application.

### BRIEF DESCRIPTION OF THE DRAWINGS

FIG. 1 shows a schematic of various problems that may cause and/or accompany a skin condition to be treated according to various embodiments of this invention.

FIG. 2 shows a graph depicting the inhibitory activity of dipalmitoyl hydroxyproline and glycine soja when used in connection with certain methods described by this invention.

FIG. 3 shows a graph depicting the inhibitory activity of a glycine soja formulation prepared with Preregen for trypsin.

FIG. 4 shows a graph depicting the inhibitory activity of a dipalmitoyl hydroxyproline for trypsin and elastase.

### DETAILED DESCRIPTION OF CERTAIN EMBODIMENTS

This invention relates to formulations for treating or preventing certain skin conditions, such as diaper dermatitis and other conditions caused by prolonged contact of the skin with proteolytic enzymes, particularly those present in human waste. The formulations include an effective amount of at least one enzyme inhibitor that at least partially inhibits enzyme activity. The phrase “effective amount” means an amount of the composition of the present invention which, when applied to an area of the body, will be effective to at least partially inhibit activity of fecal and urinary enzymes.

The inventor has found that certain compounds that are marketed as cosmetic compounds intended to protect against irritation and accelerated skin aging are unexpectedly useful for treating diaper dermatitis and other skin conditions. In order to fully describe the inventor's development, it is helpful to understand how anti-aging compounds work.

Regular exposure to sunlight, harsh conditions (such as wind, extreme temperatures and pollution), cigarette smoking, stress, or simply the passing of years takes its toll on skin. Skin loses its elasticity due to breakdown of collagen fibers, develops wrinkles and loses moisture. Accordingly, the cosmetics industry has sought to develop compounds that help fight these conditions.

Skin is composed of collagen, a protein that polymerizes to form a three-dimensional network of fibers throughout the dermis. Fibroblasts bind to the collagen fibers and cross-link them to generate a strong, stable network. Over time, the points at which cells are attached to the matrix eventually weaken, reducing the ability of fibroblasts to contract the collagen fibers, and resulting in the skin's loss of firmness.

Elastin is another fiber-building protein that makes the skin elastic. It gives skin the capacity to stretch in response to a force and then return once the force has been withdrawn. Elastin polymers are held together by bonds that are formed between amino acid residues present in peptide chains. The flexibility of the skin depends on this heavily cross-linked network of elastic fibers.

Elastases are enzymes produced by the body's own skin cells. Normally, elastase activity is regulated so that it ensures steady catabolism and turnover of elastin to prevent elastosis problems due to hardening of the dermis. However, with increasing age, the normal pattern of inhibition and activation of these enzymes is reversed, and elastase activity may be turned on inappropriately, destroying most of the bonds between different elastin molecules and causing a disruption in the elastic fibers network. Accordingly, some areas of the cosmetic industry have researched ways to inhibit these enzymes so that they do not break down the skin bonds.

The inventor unexpectedly has found, however, that certain compounds used by the cosmetics industry to fight aging are useful for treating diaper dermatitis. While the cosmetics field focuses primarily on protecting the skin from damage from the environment or from elastases produced by the cells, it is not concerned with protecting the skin from digestive or proteolytic enzymes, which are enzymes with a quite different activity from the activity of elastases. However, the present inventor has found that certain compounds used by the cosmetics industry are useful in preventing skin conditions caused by the digestive enzymes present in feces and urine that attack skin during prolonged exposure.

For example, one embodiment of the invention is directed to formulations that comprise glycine soja (soybean) protein. The formulations may also optionally include oxidoreductases. Without wishing to be bound to any theory, the inventor believes that glycine soja (soybean) protein is useful for treating diaper dermatitis because due to its structure, it competes for the active site of the protease. Since the active site of most serine proteases (especially the fecal serine proteases trypsin, chymotrypsin and elastase) share some homology and have highly conserved amino acid residues, it is believed that Glycine soja proteins inhibit members of the serine protease family showing homology at the active center, and thus, inhibit the fecal serine proteases which are associated with skin breakdown due to incontinence.

One specific form of this compound, marketed by Centerchem, Inc., is Preregen®. Preregen® is a mixture of natural oxidoreductases (which are capable of neutralizing oxygen free radicals) and isolated plant-peptides (such as glycine soja peptides) which protect the skin against environmental aggressors, such as ozone (O₃), UV radiation and detergents. It is preserved using 0.5% Phenonip® (parabens, such as methylparaben, butylparaben, ethylparaben, and propylparaben in phenoxyethanol). Additional solvents that may be included in the formulation are water and glycerin. Prepregen® formulations can remain stable for at least one to two years if properly stored.

The combination of compounds in Preregen® has been found to protect against accelerated skin aging. Skin is increasingly exposed to harmful substances present in the air; ozone is one of the most aggressive atmospheric pollutants. It can destroy the barrier function of the skin by lipid peroxidation and thus trigger irritation reactions. Because of this, researchers in the cosmetic field have sought to develop cosmetic products that inactivate oxygen free radicals and inhibit harmful proteinases in order to provide a protective shield that combats accelerated aging.

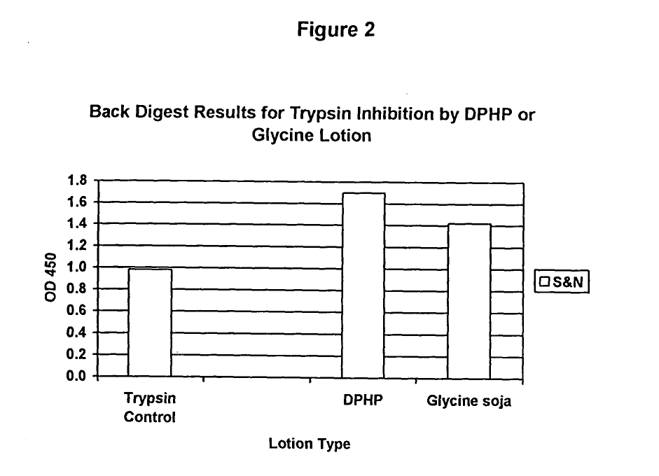

In contrast to this use, however, the inventor has found that a glycine soja (soybean) protein composition can protect skin from diaper dermatitis. In particular, as shown by FIG. 2, glycine soja protein has particular activity as a trypsin inhibitor.

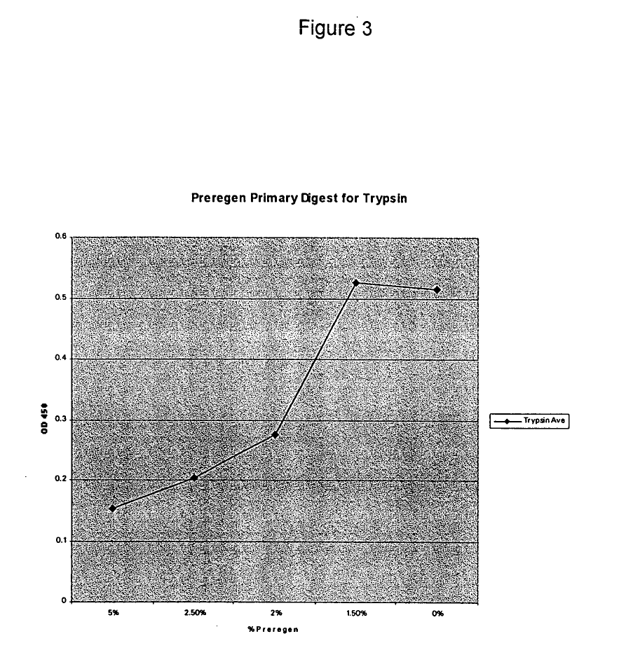

FIG. 3 shows a concentration curve for a Glycine soja protein composition. It shows concentration-dependent inhibition of trypsin activity. Glycine soja solutions at various concentrations were incubated with a gelatin-albumin matrix containing a dye. As the trypsin digested the albumin, the dye was released. The dye release was quantified via spectrophotometric analysis. The more dye released, the higher the spectrophotometric value, and the higher the trypsin activity. Therefore, in this assay, a lower reading suggests inhibition of trypsin activity. As seen in FIG. 3, a Glycine soja concentration of between 2 to 5% was found to effectively inhibit the trypsin reaction. Thus, when combined with a polymer that can bind the protein to the skin, glycine soja protein can act as an effective treatment or prevention for diaper dermatitis.

The glycine soja (soybean) protein/oxidoreductase formulation of the present invention differs significantly from the soybean inhibitors (STI) that have been studied and described by literature in the past. For example, soybean trypsin inhibitor (STI) is a low molecular weight isolate from soybean. It ranges from approximately 20-25 kD. It has been most widely studied with respect to trypsin inhibition, and its optimum pH (for activity) is 6.5.

Trypsin-chymotrypsin inhibitor is a soybean inhibitor (but not soybean trypsin inhibitor). It is a low molecular weight (Bowman-Birk type inhibitor), ranging from about 3-6 kD. It is a non-specific serine protease inhibitor which can simultaneously inhibit trypsin and chymotrypsin, and is known as being one of the more expensive inhibitors.

By contrast, Preregen® is a synergistic combination of soybean-based peptides with biotechnologically prepared natural oxidoreductases. Its molecular weight ranges; the soy component has a range of about 3-30 kD and the oxidoreductase portion is about 35 kD. The soy protein shows optimum activity between pH 5.5-6. The inhibitor of this invention is also effective against free radicals (which are formed in contact dermatitis and diaper rash is a form of contact dermatitis). On the other hand, STI is not expected to be effective against free radicals.

Additionally, STI is a Kunitz type protease inhibitor (referring to its mode of action), trypsin-chymotrypsin inhibitor is a Bowman-Birk type, while Preregen® (due to its molecular weight range) is a combination of soy proteins, some of which are Kunitz type inhibitors and some of which are Bowman-Birk type inhibitors. Therefore, it is a significant improvement over using simply a Kunitz or a Bowman-Birk type inhibitor alone.

Thus, although Preregen® and STI's or soybean inhibitors each have a soy component, they exhibit very different activities. One of ordinary skill in the art would expect them to have different inhibitory effects on the various proteases.

An alternate embodiment of the invention is directed to a formulation that comprises dipalmitoyl hydroxyproline (also referred to as “DPHP”). Dipalmitoyl hydroxyproline can be derived by grafting a fatty acid (palmitic acid) onto an amino acid (hydroxyproline) to create dipalmitoyl hydroxyproline. Without wishing to be bound to any theory, the inventor believes that dipalmitoyl hydroxyproline is useful for treating diaper dermatitis because of its anti-proteolytic activity. Dipalmitoyl hydroxyproline is a competitive inhibitor for the active site of elastase. Fecal serine proteases (trypsin, chymotrypsin and elastase) all share some homology and accordingly, the present inventor has found that the dipalmitoyl hydroxyproline will be inhibitory to all 3 of these proteases.

One form of dipalmitoyl hydroxyproline is plant-derived dipalmitoyl hydroxyproline obtained from Seppic and marketed as Sepilift® DPHP. Sepilift® is marketed as stimulating the remodeling and contraction of collagen fibers, protecting elastic fibers against enzymatic breakdown, and scavenging free radicals. It has been shown to inhibit the over-production of the elastase enzymes that destroy bonds between different elastin molecules of the skin.

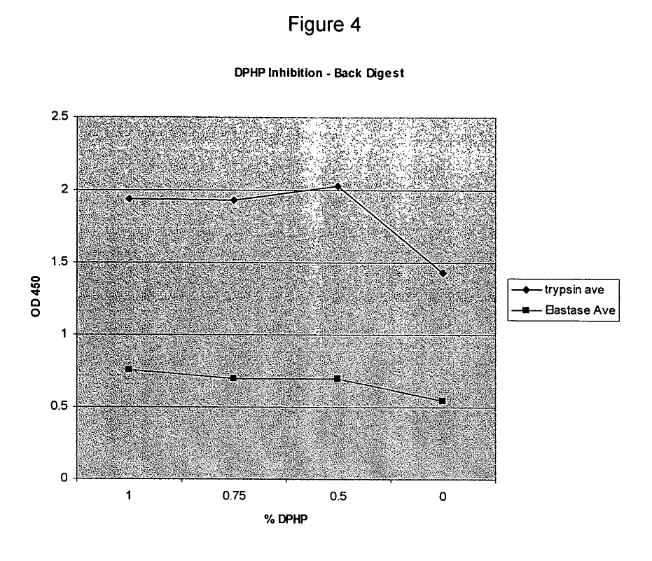

The inventor has found, however, that in addition to inhibiting the skin's natural production of elastases, dipalmitoyl hydroxyproline also inhibits the elastases and proteases (e.g., trypsin and chymotrypsin) found in feces and urine. As shown by FIGS. 2 and 4, DPHP inhibits trypsin and elastase, which are digestive enzymes that are present in human waste that can attack the skin's integrity. The results from FIG. 2, indicate that a lotion made with DPHP provided significant reduction in trypsin activity. FIG. 4 indicates that the DPHP material itself is inhibitory to both elastase (−22%) and trypsin (−33%) activity (higher back digest values indicate lower protease digestion of substrate).

Sepilift® DPHP is quite a different molecule from any of the inhibitors that have previously investigated in connection with diaper dermatitis. Studies by the manufacturer (Seppic) show that DPHP is a potent inhibitor of elastase. However, this product is not considered in the group of classical serine protease inhibitors. There is no indication in the literature that it would be an effective inhibitor of trypsin or chymotrypsin. However, the inventor has determined that DPHP does inhibit trypsin, elastase and chymotrypsin, and as such, can be used to treat or prevent diaper dermatitis or other associated skin conditions.

It is important to note that DPHP is also different from classical serine protease inhibitors in that the manufacturer has shown it to be a potent inhibitor of free radicals. (Both Preregen® and DPHP share this free radical inhibitory effect.) Finally, DPHP has a molecular weight of 450 daltons. It is smaller than the plant-derived serine protease inhibitors.

Previous compositions which incorporated some form of enzyme inhibitor may have side effects if absorbed by the skin. By contrast, the inhibitors of this invention are currently used in the cosmetic market for anti-aging and are generally recognized as safe and without side effects. The enzyme inhibitors used are specifically targeted by the vendors as elastase inhibitors. Elastase breaks down the elastin (which is the protein that provides skin with both its elasticity and strength) in the skin.

However, contrary to what is commonly known about glycine soja (soybean) protein or dipalmitoyl hydroxyproline, the inventor has found that they are also potent inhibitors of trypsin and chymotrypsin, both of which are involved in skin breakdown due to incontinence. These enzyme inhibitors have also been shown to counteract the effects of free radicals, which can contribute to diaper dermatitis. Any tissue injury capable of producing an inflammatory response, diaper dermatitis included, will have an important oxidative (free radical) component and as such, will benefit from the addition of a free radical inhibitor. Free radicals may significantly contribute to skin breakdown and are known to be involved in inflammatory responses. Free radicals are also found in higher amounts in elderly people, who are more at risk for incontinence.

Compositions according to certain aspects of this invention may also include a sacrificial lipase inhibitor. Lipase in combination with other fecal proteases is a potent mediator of skin breakdown. Particularly, lipases are found in feces and are known to contribute to skin breakdown and skin irritation due to incontinence when in combination with serine proteases. Thus, a fatty acid such as coconut oil or any other any other hydrolysable fat such as sunflower oil, castor oil, almond oil or similar oil may be included to act as a sacrificial substrate for lipase.

The composition may also include a sacrificial substrate for proteases in general, for example, an amino acid that will inactivate proteases with which it comes into contact. Possible sacrificial substrates include but are not limited to soy amino acids, denatured and native proteins such as gelatins, denatured collagens, and similar proteins.

Additional embodiments of the invention may also include a pH buffering system. Fecal enzymes are activated at the elevated pH's which can be found in urine. The buffering system is used to maintain the pH of not only the product, but the skin at a pH of about 5.5-6.5. This is beneficial because proteolytic enzymes are less active at lower pH values (and show higher proteolytic activity above pH 7). The type of buffering system may include a phosphate ion buffer combination, a lactic acid/lactate system, or any other appropriate buffering system.

The compositions also include a topical polymer that is capable of binding the enzyme inhibitors and holding them in close contact with the skin for prolonged periods of time. This is an important aspect of certain embodiments of the invention. The polymer should adhere to the outer skin layer and form a protective barrier that is substantially resistant to being washed or rubbed off. It should also be substantially resistant to certain chemical insults, particularly enzymatic insults. Particularly useful polymers also reduce skin irritation and dryness. Particularly suitable polymers are polymers in the M or D series from Skinvisible®.

The Skinvisible® polymers are adapted to adhere to a substrate, such as a patient's skin, and form a protective bond. They are substantially resistant to being washed or rubbed off, and deliver a targeted level of therapeutic or cosmetic agent to the skin. They are manufactured to provide an efficient, long-lasting dose without irritation.

The M-series polymers comprise primarily maleic acid methylvinyl-ether copolymer. They form a bond with the skin that resists wash off and that is non-occlusive. The polymer is soluble at about 65° C. and maintains a pH range between about 5.5-8.0. They have been used in connection with antimicrobials, sunscreens, antifungals, cosmetics, and moisturizers. They have not been used in connection with a diaper rash treatment.

The D-series polymers comprise primarily dextran-70. They form stable emulsions and maintain a pH range between about 4.0-8.0. They have been used to encapsulate volatile active ingredients. They have not been used in connection with a diaper rash treatment.

The Skinvisible® polymers are particularly useful as the delivery system because they are elegant to the touch and are non-greasy. They also bind to the skin and provide a continuous layer in the form of a protective barrier which is long lasting and resistant to wash off. The protease inhibitor used in the formulation is bound to this barrier and thus remains on the skin surface. Thus, the formulation not only allows the polymer itself to inhibit the contact of noxious proteases with the skin surface, but it also holds and allows the protease inhibitor to inhibit the harmful proteases. Thus, even if the barrier (polymer) became compromised, the proteases are likely negated by the inhibitors before they can even act on the skin.

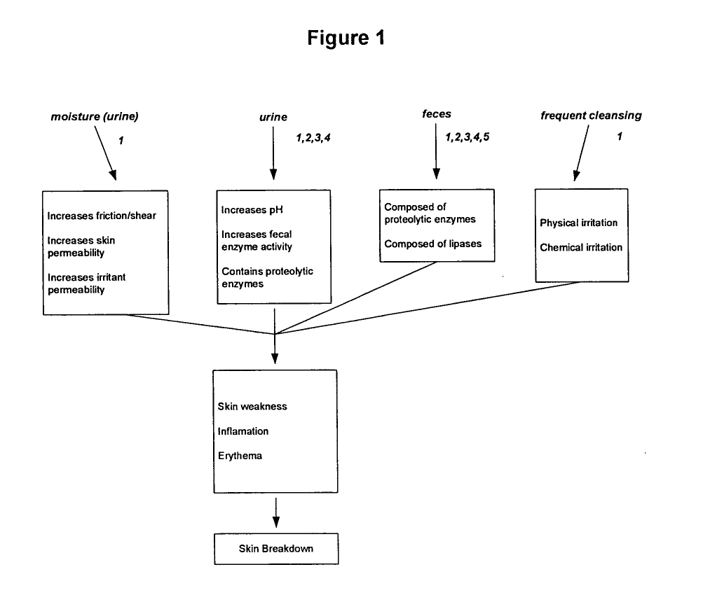

The stages of skin breakdown due to incontinence are shown in FIG. 1. The numerals indicate various stages where the different combative agents that are included in formulations according to various embodiments of this invention would operate. For example, one formulation includes a polymeric system that includes an enzyme inhibitor, a buffering system, a sacrificial protease substrate and a sacrificial lipase substrate. This embodiment would likely have its components effective at the following numerals of FIG. 1: 1—polymeric system; 2—buffering system; 3—enzyme inhibitor; 4—sacrificial protease substrate; and 5—sacrificial lipase substrate.

Compositions according to various embodiments of this invention may take the form of a cream, ointment, lotion, gel, foam, spray, liquid, or any other topical application method. In some embodiments, particularly the cream or lotion embodiments, the formulation includes a thickening and stabilizing system of stearic acid, cetyl alcohol and a polymeric or carbomeric system such as carboxymethylcellulose, microcrystalline cellulose and cellulose gum, carrageenans, sodium polyacrylate or carbomer 934. This system may be replaced with any other thickening system which is stable with the use of the aforementioned ingredients, such as other carbomeric and polymeric combinations, microcrystalline cellulose, and the like.

Alternatively or additionally, the current compositions may include glyceryl monostearate as a gentle emulsifier. Other gentle emulsifiers may be used including nonionic emulsifiers, such as polysorbates, and nonionic glucolipids, such as a cetearyl alcohol and cocoglucoside combination.

Various compositions of this invention may also include a monographed skin protectant such as dimethicone or allantoin, colloidal oatmeal, or any other monographed ingredient from 21 CFR Parts 310, 347 and 352 which is deemed appropriate. (These may include monographed items considered appropriate as anorectal or anti-itch drugs. Monographed ingredients refer to those allowable, OTC, drug product ingredients for skin protectant use which are generally recognized as safe by the FDA.)

The current composition may also include an odor neutralizer, such as zinc rinoleate or ordenone®, or any other appropriate odor neutralizer. Additionally, it may include one or more other skin health benefit agents, such as stearic acid, isoparrafin, petrolatum, fatty acids, fatty acid esters, triglycerides, phospholipids, mineral oils, essential oils, sterols, sterol esters, emollients, moisturizers, waxes, humectants, surfactants, anti-inflammatories, amino acids such as soy or oat amino acids, anti-microbials, anti-puretics, hydroxy acids, microbial or algal extracts, anti-histamines, anti-oxidants, analgesics, astringents, fragrances, dyes, natural or synthetic vitamins, and/or deodorants, examples of which appear in WO 00/38747, the contents of which are incorporated here by reference.

Examples of excipients that may be included with formulations are polymers of a carboxyvinyl type, polyethyleneglycols, propyleneglycol, waxes, fatty substances, esters and triglycerides of fatty substances, stearic derivatives such as, for example, glycerol stearate, alcohols such as, for example, stearyl alcohol, ketostearyl alcohol, ketyl alcohol, polyol, polyoxyethylene ketyl ether, vegetable oils such as soft almond oil, coconut oil, castor oil, mineral oils such as Vaseline oil, glycerin, derivatives of lanolin, talc, wetting agents, thickening agents, stabilizing agents, emulsifying agents, preservatives, perfumes, colorants or other known and currently used excipients.

Additional ingredients which may be present in the copolymer composition include oils such as mineral oil, fish liver oil, and cod liver oil; emollients such as glycerin, olive oil, and lanolin; fillers such as cellulose gum, calcium carbonate, karaya gum, gum tragacanth, gum acacia, carboxymethyl cellulose, and polyvinyl acetate; vitamins such as vitamins A, D, and D3; astringents such as zinc oxide and aluminum acetate; protectants such as Peruvian balsam; coloring agents; odorants; and other materials which are conventionally used in relieving skin irritation.

This invention is also directed toward methods of preparing compositions according to certain embodiments of this invention. One method for the formulation of the composition of this invention is to add the polymeric system from Skinvisible® to the enzyme inhibitor (either glycine soja (soybean) protein or dipalmitoyl hydroxyproline), sacrificial protease substrate and sacrificial lipase substrate. A gentle emulsifier, typically glyceryl monostearate, and a stabilizing system, typically stearic acid and cetyl alcohol are added. The mixture is heated to an appropriate temperature and water, preservatives, and thickener are added.

The current polymeric protective barrier and protease inhibitor composition is a significant improvement over previous polymeric barriers, dimethicone and zinc oxide (commonly used in diaper rash preparations). The current formulation binds strongly to the skin for hours and only begins to come off the skin via natural exfoliation. The polymer also binds very strongly to hydrophilic and hydrophobic actives. Thus, the combination of polymer plus the enzyme inhibitor will be held at the skin surface for long periods of time where it can exert its effect on the proteolytic enzymes of the feces and urine.

Where many barrier products are sticky and hard to remove during cleaning, the current formulation is non-greasy, transparent (or substantially transparent), and will allow for easy cleaning after an incontinent episode. Since scrubbing is not required as it is for some thick, messy barrier creams, the present formulation also helps decrease the chemical and physical irritation caused by cleaning. In other words, the emulsified, aqueous-based products of this invention are easy to apply and remove, and the tissue trauma associated with difficult ointment removal is avoided. The products are elegant to the touch, and allow visual inspection of the skin whereas some ointments and barriers are opaque and prevent visual skin inspection.

Certain embodiments of polymers used in connection with this invention are also anti-irritants and have been shown to have moisturizing properties. This is beneficial because although incontinence increases the wetness of the skin, many incontinent patients' skin actually lacks moisture. Proper moisturization is critical to skin health. As the skin dries, it may crack and become a suitable environment for bacterial growth. Accordingly, it is desirable to provide a product that enhances the skin's moisture, and possibly to add additional moisturizers to the formulation. Thus, the current polymer will also benefit the patient by increasing or maintaining skin moisturization.

### EXAMPLE 1

An experiment was conducted to examine the inhibitory effect of a formulations prepared according to this invention (with coconut oil) on lipase inhibition. Whole milk (containing emulsified fat) was mixed with bile salts to more fully disperse the fat. This mixture was then placed in a test tube with a lipase solution or a lipase solution+prototype formulation and pH was monitored over time. As fatty acids were liberated from the milk via lipase digestion, the pH of the milk solution decreased. The initial reaction rate or pH decrease was calculated over time.

Table 1 indicates that the initial reaction rate is highest without the formulations of this invention added, which means that the anti-lipase activity of formulations according to this invention provide added protection from skin breakdown due to the presence of incontinence related enzymes. In other words, when the Glycine soja protein (in this case, Preregen) or DPHP formulations are added, the initial reaction rate is decreased. For the Preregen formulation the rate decrease was 32% and for the DPHP formulation the rate decrease was 93%.

TABLE 1
Inhibition of Lipase Activity by Preregen and DPHP formulations

| | Initial Reaction Rate | |
|---|---|---|
| Condition | (Δ pH units per min) | % Inhibition |
| Control (Milk + bile salts + lipase) | 0.28 | |
| Milk + bile salts + Preregen | 0.19 | −32% |
| formulation | | |
| Milk + bile salts + DPHP formul- | 0.02 | −93% |
| ation | | |

### EXAMPLE 2

The inventor selected glycine soja and DPHP by testing numerous potential inhibitors in vitro to assess their activity against protease enzymes. A protease enzyme present in feces or urine, such as trypsin, chymotrypsin, or elastase, was placed in contact with a substrate (crosslinked albumin, sulfaniloazo-albumin and gelatin). Separately, the protease (trypsin, chymotrypsin or elastase) was incubated with a protease inhibitor and then this combination was placed in contact with the substrate. The substrate-protease or substrate-protease-protease inhibitor combination was then left to incubate.

At a defined time, the incubation was stopped by the addition of NaOH. Color was developed and read at OD 450. The intensity of the color developed was directly proportional to the amount of substrate digested by the protease. This allowed the inventor to analyze which protease inhibitors prevented the most substrate digestion.

### EXAMPLE 3

FIG. 2 shows the effectiveness of both a DPHP prototype and a glycine soja prototype for inhibiting the protease, trypsin. A back digest was performed with a spectrally active protease substrate, trypsin, and either DPHP or glycine soja. Trypsin was incubated with either DPHP or glycine soja, and for the control, neither DPHP nor glycine soja was used. The trypsin or trypsin+either DPHP or glycine soja was then placed in contact with a substrate, the results of which are shown in FIGS. 3 and 4.

The substrate used was a crosslinked albumin, sulfaniloazo-albumin and gelatin at a slightly acidic pH. The substrate is susceptible to proteolysis by a wide range of enzymes including collagenase, papain, trypsin, chymotrypsin and bromelain. Digestion of this substrate by protease releases a dye. Color is developed at the end of the reaction period via the addition of 0.1 N NaOH. This color development can be measured spectrophotometrically at OD 450 and the OD value is proportional to enzymatic activity. In a typical digest, enzyme or enzyme+inhibitor are placed in contact with substrate and the reaction is allowed to proceed at 37° C. for a set time period. After the initial digest, color is developed by the addition of NaOH. The OD 450 reading is directly proportional to the enzyme activity (i.e. the higher the value, the higher the enzyme activity).

### EXAMPLE 4

A back digest was conducted when particulate suspensions were used as either enzyme or enzyme inhibitor or when lotions were being tested, because the particulate interferes with the OD readings. FIGS. 2 and 4 show the results of back digest tests that were conducted. The enzyme or enzyme+enzyme inhibitor was placed in contact with the substrate for a set amount of time. After this digest, the solutions were poured off, the substrate was washed and an excess of enzyme was placed in contact with the substrate for an additional incubation period. At the end of this incubation, the color was developed as described above, with the addition of 0.1 N, NaOH. Color development was read at OD 450. The color was then compared with controls which had no enzyme. For back digests, higher values are inversely proportional to enzyme activity. Thus, higher values are indicative of increased enzyme inhibition.

After an initial digest, the supernatant was decanted, and an excess of trypsin was added for further digestion of the substrate. After this final digest, supernatant optical density (OD) was read at 450 nm. (Higher OD values (above control) indicate that more substrate remained after the initial digest, and as such, that trypsin activity was inhibited.)

### EXAMPLE 5

FIG. 3 shows a concentration curve for a Glycine soja protein composition. It shows concentration-dependent inhibition of trypsin activity. Glycine soja solutions at various concentrations were incubated with a gelatin-albumin matrix containing a dye. As the trypsin digested the albumin, the dye was released. The dye release was quantified via spectrophotometric analysis. The more dye released, the higher the spectrophotometric value, and the higher the trypsin activity. Therefore, in this assay, a lower reading suggests inhibition of trypsin activity. As seen in FIG. 3, a Glycine soja concentration of between 2 to 5% was found to effectively inhibit the trypsin reaction. Thus, when combined with a polymer that can bind the protein to the skin, glycine soja protein can act as an effective treatment or prevention for diaper dermatitis.

### EXAMPLE 6

Using the above methods, two protease inhibitors mentioned by the prior art were tested. A stabilized preparation of hexamidine (Elestab) and another preparation of 4-(2-aminoethyl)-benzenesulfonylfluoride hydrochloride (AEBSF) initially showed very high protease inhibition activity. However, they were left for one month and then tested for activity. After one month at room temperature, the activity of the stabilized AEBSF solution was decreased by 42%. When a sample of Elestab solution was left at room temperature for a month, its inhibitory activity was shown to decrease by about 37% over a freshly prepared Elestab solution.

These results for AEBSF correspond to those previously found for aqueous solutions of enzyme inhibitors. The half life for many highly reactive inhibitors (such as AEBSF) was from hours to days. Indeed, the solution lifetime of many reactive inhibitors such as AEBSF and a number of others can be quite short due to their highly reactive nature. In the presence of water they tend to hydrolyze.

Accordingly, although the inhibitors that have been studied previously may initially have high anti-protease activity, this activity is short lived in aqueous environments. Therefore, many of the inhibitors of the prior art are unsuitable for inclusion into an emulsion such as a lotion, cream or spray.

The present invention solves this problem by providing an improvement over these technologies since the inhibitors used in connection with this invention have been shown to be stable over time. Indeed, lotions prepared with DPHP and Preregen® retained their activity over a number of months.

Accordingly, the combination of the novel polymer barrier bound to a protease inhibitor such as glycine soja or DPHP and applied to the skin provides the significant improvement over prior art. In certain embodiments, the type of delivery vehicle used will be important in keeping the inhibitor in contact with the fecal proteases and away from the skin. In those embodiments, the addition of a polymer, such as an M or a D series Skinvisible® polymer, is an important aspect of the invention. The features of the preferred polymers are that they should be non-sticky, long lasting and substantially resistant to being washed or rubbed off.

The particular embodiments of the invention described above are merely illustrative and are not the only embodiments possible. Those skilled in the art can readily identify additional embodiments and features of the invention that are within the scope of the appended claims.

1. A protease inhibitor composition, comprising:
a polymer barrier and a protease inhibitor, wherein the polymer barrier binds the protease inhibitor to a patient's skin and is substantially resistant to being washed or rubbed off.

2. The protease inhibitor composition of claim 1, wherein the polymer barrier comprises a Skinvisible® M-series or D-series polymer.

3. The protease inhibitor composition of claim 1, wherein the protease inhibitor comprises a glycine soja protein.

4. The protease inhibitor composition of claim 3, wherein the protease inhibitor further comprises oxidoreductases.

5. The protease inhibitor composition of claim 1, wherein the protease inhibitor comprises a dipalmitoyl hydroxyproline.

6. The protease inhibitor composition of claim 1, wherein the composition has an extended shelf-life.

7. A protease inhibitor having an extended shelf life for use in treating diaper dermatitis-like skin conditions comprising a cosmetic protease inhibitor that maintains its activity in emulsion for at least about one year.

8. The protease inhibitor composition of claim 7, wherein the cosmetic protease inhibitor comprises a glycine soja protein or dipalmitoyl hydroxyproline.

9. The protease inhibitor composition of claim 7, wherein the cosmetic protease inhibitor comprises a combination of glycine soja protein and oxidoreductases.

10. The protease inhibitor composition of claim 7, further comprising a sacrificial protease substrate.

11. The protease inhibitor composition of claim 7, further comprising a sacrificial lipase substrate.

12. The protease inhibitor composition of claim 7, further comprising a polymer barrier.

13. The protease inhibitor composition of claim 12, wherein the polymer barrier comprises a Skinvisible® M-series or D-series polymer.

14. A cosmetic protease inhibitor that is suitable for delivery as an emulsion for use in treating diaper dermatitis.

15. A method of treating diaper dermatitis comprising applying a composition comprising glycine soja protein to an affected area of a patient in need thereof.

16. A method of treating diaper dermatitis comprising applying a composition comprising dipalmitoyl hydroxyproline to an affected area of a patient in need thereof.

17. A method of formulating a diaper dermatitis treatment, comprising
(a) providing a polymeric system that can bind to a patient's skin;
(b) combining the polymeric system with glycine soja protein or dipalmitoyl hydroxyproline;
(c) adding a sacrificial protease substrate;
(d) adding a sacrificial lipase substrate;
(e) adding a pH buffering system; and
(f) forming the mixture into a cream or a lotion for topical application.
